(19) United States
(12) Patent Application Publication
(10) Pub. No.: US 2011/0112287 A1
(43) Pub. Date: May 12, 2011

(54) CARBON NANOPARTICLES, WHICH ARE COVALENTLY BOUND VIA A BRIDGE MOLECULE TO A TARGET MOLECULE, AND A METHOD FOR THE PRODUCTION THEREOF

(75) Inventors: Teodor Silviu Balaban, Karlsruhe (DE); Stefanie Potratz, Pforzheim (DE); Frank Hennrich, Karlsruhe (DE); Regina Fischer, Karlsruhe (DE); Michaela Carmen Balaban, Karlsruhe (DE); Manfred Kappes, Ettlingen (DE)

(73) Assignee: KARLSRUHER INSTITUT FUER TECHNOLOGIE, Karlsruhe (DE)

(21) Appl. No.: 12/912,892

(22) Filed: Oct. 27, 2010

(30) Foreign Application Priority Data
Oct. 28, 2009 (DE) 10 2009 051 126.1

(51) Int. Cl. C09B 47/04; C07C 63/42; C07C 69/76; C07C 313/02; C07C 63/00; C07F 7/04; C07D 487/22; C07D 303/12; B82Y 30/00; B82Y 40/00; B82Y 5/00

(52) U.S. Cl. 540/122; 562/492; 560/102; 558/61; 562/887; 556/482; 540/145; 549/512; 977/773; 977/847; 977/896; 977/742; 977/788; 977/904; 977/948; 977/915; 977/932

(57) ABSTRACT

A method for covalently binding target molecules to carbon nanoparticles via at least one bridge molecule includes converting carbon nanoparticles to acyl carbon nanoparticles using a carbonyl compound of the at least one bridge molecule in the presence of a Lewis acid under Friedel-Crafts conditionsm, where the acyl carbon nanoparticles include a nucleofuge in the omega position. The target molecule is covalently bound to the acyl carbon nanoparticles via nucleophilic substitution of the nucleofuge in the omega position.

This application claims priority to German Patent Application No. DE 10 2009 051 126.1, filed on Oct. 28, 2009, which is hereby incorporated by reference herein in its entirety.

### FIELD

The present invention relates to modified carbon nanoparticles, in particular, modified carbon nanotubes, which are bound via bridge molecules (spacers) to target molecules (materials).

### BACKGROUND

Carbon nanoparticles are generally known. Carbon nanotubes constitute one form of carbon nanoparticles. Carbon nanotubes have very small cylindrical structures having a diameter of one to a few nanometers and an aspect ratio of 10 to 1000. Carbon nanotubes have a honeycomb-type hexagonal structure in which each carbon atom is bonded to three adjacent carbon atoms. Depending on their precise structure, carbon nanotubes can act as electrical conductors or as semiconductors. Carbon nanotubes can be in the form of what are generally referred to as single-walled carbon nanotubes (SWNT), for example. For the sake of simplicity, these carbon nanotubes are also referred to as SWNT in the following. Their unique properties also make these SWNT the focus of intense research. Strength, resistance, rigidity, a very high modulus of elasticity, as well as thermal and electrical conductivity count among the properties of the SWNT.

SWNT are essentially composed of sp² hybridized carbon atoms, which, according to B. I. Yakobsen and R. E. Smalley, American Scientist, vol. 85, July-August, 1997, 324-337, are typically arrayed in pentagons or hexagons. These can be produced in larger quantities and in a reproducible quality by a controlled growth that is catalyzed by metal nanoparticles. In addition, the carbon nanotubes can also exist in the form of what is generally referred to as multiwalled carbon nanotubes (MWNT). MWNT are SWNT that are concentrically nested within one another and that have properties which are similar, but inferior to those of the SWNT. In contrast to the MWNT, the SWNT have fewer defects and, accordingly, are stronger, more resistant and have a higher conductivity.

Whether a particular carbon nanotube is metallically conductive, semiconductive or non-conductive is determined, inter alia, by its chirality. A classification by diameter is likewise possible, SWNT having a diameter of 0.7 to 3 nm and MWNT a diameter of 2 to 20 nm.

To be able to utilize the advantages and special properties of carbon nanoparticles, a binding to a target molecule or a material is required. There are only few examples known from the technical literature where carbon nanotubes are covalently bound to materials. This is due to the difficulty entailed in chemically modifying carbon nanotubes. Due to the strong vander-Walls mutual forces of attraction of the carbon nanotubes and the high aspect ratio, carbon nanotubes exist in agglomerates and are thus insoluble in most solvents. A homogeneous dispersion of carbon nanotubes is impeded by this tendency to exist in agglomerates. Therefore, the singularization and dispersibility of carbon nanotubes are important points to consider when processing the same in mixtures with other materials.

Ongoing research is directed to the processing of carbon nanotubes in polymer composites (for example, epoxy resins). Most of the strategies deal with increasing dispersibility, including ultrasound treatments, intensive mixing under shear force, admixing tensides, chemical modification by functionalization, using polymers to sheathe the carbon nanotubes, and various combinations thereof. Only few methods are known for covalently binding carbon nanotubes to the epoxy resin.

Carbon nanoparticles have also been investigated in pharmaceutical research as what are generally referred to as transfection agents. Due to their property whereby they are capable of traversing cell membranes, carbon nanotubes are of interest as carriers for biologically active substances, such as peptides, nucleic acids and pharmacological agents.

The binding of carbon nanotubes to materials is a time-consuming, laborious and cost-intensive process. Neutral carbon nanotubes cannot be bound directly to materials. Chemical modifications using useful functional groups are necessary.

The articles by D. Tasis, N. Tagmatarchis, A. Bianco, M. Prato Chem. Rev. 2006, 106 (3), 1105-1136 and X. Peng, S. S. Wong Adv. Mater. 2009, 21, 625-642 describe methods for oxidizing carbon nanotubes using a mixture of fuming nitric acid and concentrated sulfuric acid. These oxidized carbon nanotubes exhibit an improved solubility. The presence of organic acid groups makes possible a multiplicity of additional modification options using widely diversified materials. For example, the acid can be converted to esters or amides through the use of thionyl chloride and alcohols or amines. Dyes with alcohol groups were covalently bound under esterification conditions to what are generally referred to as SWNT acids. The direct esterification or amination by alcohols or amines under the catalysis of diimides is also described.

J. Zhu, J. Kim, H. Peng, J. L. Margrave, V. N. Khabashesku, E. V. Barrera Nanolett 2003, 3(8), 1107-1113 and D. Tasis, N. Tagmatarchis, A. Bianco, M. Prato Chem. Rev. 2006, 106 (3), 1105-1136 describe a multi-stage synthesis of covalently functionalized carbon nanotubes that renders possible the binding of SWNT to epoxy resins. The ends of the SWNT were first oxidized in a mixture of nitric and sulfuric acid. Following fluorination of the thus purified and modified SWNT, these were successfully covalently functionalized by the nucleophilic substitution of the fluorine atom with amines or diamines. The SWNT functionalized in this manner were blended with epoxy resin. Thus, the epoxide reacts both with amines, as well as with the acid groups from the oxidation.

The article by Zhu et al. (J. Zhu, H. Peng, F. Rodriguez-Macias, J. L. Margrave, V. N. Khabashesku, A. M. Imam, K, Lozano, E. V. Barrera Adv. Funct. Mater. 2004, 14 (7), 643648) describes a synthesis pathway whereby SWNT that had been purified by the treatment with acid and functionalized at the ends, were subject to a radical addition. Alkyl-substituted SWNT were able to be produced in this manner. If succinic anhydride is used, the SWNT are then functionalized by ethylcarboxyl groups. The acyl groups were chlorinated with the aid of thionyl chloride and subsequently reacted with diamines. The SWNT functionalized in this manner were blended with epoxy resins in a chloroform solution and cured following removal of the solvent.

World Patent Application WO 2008104079 describes a method for reducing carbon nanotubes and for thereby making them accessible to nucleophilic substitutions, for example, with epoxy resins. In this manner, materials can be covalently bound to carbon nanotubes directly or via what are generally referred to as spacers. Bifunctional carbon nanotubes were bound to epoxy resins and other polymers, such as polystyrene, for example.

The World Patent Application WO 2008104078 describes a method for covalently binding the epoxy resin by a spacer to carbon nanotubes in a radical process in only one step by reacting the same with epoxy resin and an alkene. The opening of the double bond to a radical can be initiated thermally, cationically or photolytically, thereby permitting a reaction with the carbon nanotube and the epoxide. The length of the spacer is adjusted by the stoichiometric composition of alkene and epoxy resin. When the epoxy resin is reacted with carbon nanotubes using an alkene as spacer in only one reaction step, a chain length distribution of the spacer results. Thus, the length of the spacer cannot be precisely adjusted. However, directly reacting carbon nanotubes with epoxy resins is undesirable, since what is generally known as a masterbatch cannot be produced in this manner. The following describes mixing a masterbatch with different epoxy resins. The term masterbatch is understood to mean additive concentrates, which are admixed in the form of polymer-bound additives with a naturally occurring preparation or a plastic (raw polymer) for staining or for modifying the properties.

Single-walled carbon nanohorns, which are very similar to the SWNT, were successfully modified with porphyrins in three synthesis steps (C. Cioffi, S. Campidelli, C. Sooambar, M. Marcaccio, G. Marcolongo, M. Meneghetti, D. Paolucci, F. Paolucci, C. Ehli, G. M. A. Rahman, V. Sgobba, D. M. Guldi, M. Prato J. Am. Chem. Soc. 2007, 129, 3938-3945). A simply Boc-protected (tert-butyl carbamate) diamine was first made to react in a nucleophilic substitution with the carbon nanohorn. The deprotected amine was reacted with acyl chlorides. The procedure took approximately ten days.

The German Patent Application DE 102005041378 A1 describes a method for binding acyl groups to carbon nanoparticles by Friedel-Crafts acylation. The underlying problem definition in this case is increasing the solubility or the dispersibility of the carbon nanoparticles, respectively purifying the carbon nanoparticles. There is no mention of covalently binding the particles via the acyl group to a material.

Existing methods for covalently modifying carbon nanoparticles using materials are generally very protracted and often involve a great number of reaction steps. These reactions are also rendered uneconomical by the use of moderately expensive or even chemically aggressive reagents.

### SUMMARY

In an embodiment, the present invention provides a method for covalently binding target molecules to carbon nanoparticles via at least one bridge molecule. The method includes converting carbon nanoparticles to acyl carbon nanoparticles using a carbonyl compound of at least one bridge molecule in the presence of a Lewis acid under Friedel-Crafts conditionsm, where the acyl carbon nanoparticles include a nucleofuge in the omega position. The target molecule is covalently bound to the acyl carbon nanoparticles via nucleophilic substitution of the nucleofuge in the omega position.

### BRIEF DESCRIPTION OF THE DRAWINGS

The present invention is explained in greater detail in the following with reference to exemplary embodiments and to the drawings, in which:

FIG. 1 shows the general molecular structure of an embodiment of the carbon nanotubes according to the present invention;

FIG. 2 shows acylation of a carbon nanotube according to method step 1 of an embodiment of the invention;

FIG. 3 shows nucleophilic substitution of a carbon nanotube that has been acylated by a bridge molecule, in accordance with method step 2 of an embodiment of the invention.

### DETAILED DESCRIPTION

In an embodiment, the present invention provides a method that is readily accessible and has versatile applications, for covalently binding modified carbon nanoparticles, in particular carbon nanotubes, under mild conditions via a bridge molecule (spacer) to materials. Preferably, the covalent binding of unmodified carbon nanoparticles to materials take place within a short period of time and that the product be easily purified.

In an embodiment, the present invention provides carbon nanoparticles that are bound via bridge molecules to target molecules. The bridge molecules can make it possible to increase the solubility, and respectively improve the dispersibility in preparations. Moreover, bridge molecules have the function of electronically and spatially separating the target molecule (material) from the carbon nanoparticles, in particular, carbon nanotubes.

In an embodiment, the present invention provides potential uses of carbon nanoparticles that are bound via bridge molecules to target molecules. In this case, the special properties of the carbon nanoparticles, in particular, the carbon nanotubes, in combination with the target molecules via bridge molecules can be vitally important.

A novel two-step synthesis method for covalently binding carbon nanoparticles via bridge molecules to target molecules has been discovered in accordance with embodiments of the present invention. To this end, the carbon nanoparticles are reacted in a first step under Friedel-Crafts conditions in the presence of a Lewis acid to form an acylated carbon nanoparticle, preferably, a carbon nanotube.

Friedel-Crafts acylation is a known method used for producing aromatic ketones by reacting aromatic compounds with acylating agents and Friedel-Crafts catalysts.

Friedel-Crafts catalysts are what are generally referred to as Lewis acids that are reacted equimolarly with the acylation reagent during the acylation. AlCl₃, BF₃, FeCl₃ or SnCl₄ come into consideration as Lewis acids. AlCl₃ is preferably used as a Lewis acid.

Preferably suited as acylating agents in this connection are carboxylic acid chlorides and carboxylic acid anhydrides. Since, at the same time, the acylating agent is the bridge molecule that binds the carbon nanoparticle to a target molecule, it may be beneficial that, in the omega position, the bridge molecule possess a leaving group for the nucleophilic substitution, thus a nucleofuge. Halogens are preferably used as nucleofuges. Particular preference is given to the use of bromine as a nucleofuge.

When cyclic carboxylic acid anhydrides are used, a carboxyl group in omega position is formed following the hydrolytic preparation in the acylation step. The hydroxyl group of the carboxyl group in omega position may be used directly as a nucleofuge. Maleic anhydride, glutaric anhydride, phthalic anhydride or succinic anhydride, as well as the derivatives thereof, i.e., having substituents in the alkyl chain, are used as cyclic carboxylic acid anhydrides. Particular preference is given to the use of glutaric anhydride or succinic anhydride.

In the subsequent step, the nucleofuge in omega position of the acylated carbon nanoparticle is replaced in a nucleophilic substitution by a nucleophile that is already present on the target molecule. Nucleophiles are functional groups which, due to the negative or partial charge, respectively the Lewis base properties thereof, attack a group having opposite properties (electrophilic) during a reaction. Alcohols, amines or sulfides may be used as nucleophiles.

Accordingly, acylated carbon nanoparticles having a carboxyl group in omega position are reacted with the target molecule to form esters, amides or thioesters.

In an embodiment of the method, the omega-acylated carbon nanoparticles are reacted in what is generally known as a Steglich condensation at room temperature. For the esterification, the omega-acylated carbon nanoparticles are reacted with the corresponding alcohol in dichloromethane, for example, using EDC HCl (1-ethyl-3-(3-dimethylaminopropyl)carbodiimide hydrochloride) and DMAP (N,N-dimethylaminopyridine) as a catalyst. The amidation is implemented, for example, using EDC HCl and HOBt H₂O (1-hydroxybenzotriazole) as a catalyst, for example, with the corresponding amine in dichloromethane.

Provided that the nucleofuge is not bound to a carbonyl group in omega position for the acylated carbon nanoparticle, then the reaction products of the nucleophilic substitution are ethers, amines or thioethers. The omega nucleofuge-substituted carbon nanoparticles are preferably reacted in the presence of a base. As bases, those may be used which are strong enough to deprotonate the alcohols, amines or thiols. Particular preference is given to the use of potassium carbonate, caesium carbonate, potassium hydroxide, potassium or sodium tert-butanolate, potassium or sodium. In particular, sodium hydride is used.

In the context of the method according to embodiments of the present invention, the bridge molecule plays a key role since it contains a carbonyl compound that is used for binding to the carbon nanoparticles and, in addition, includes a leaving group that is replaced by a functional nucleophilic group of the target molecules. For example, when cyclic carboxylic acid anhydrides are used, the leaving group is intrinsically formed during the first reaction step.

The option for different Friedel-Crafts reagents, for example, acid chlorides and acid anhydrides, to be simultaneously used in the first reaction step also permits a mixed functionalization of carbon nanoparticles. In this case, the predefined stoichiometric compositions of the educts in the Friedel-Crafts acylation are decisive for the composition of the reaction products.

Embodiments of the present invention also encompass modified carbon nanoparticles which are covalently bound via a bridge molecule to a target molecule. The bridge molecule is bound via a carbonyl group, preferably a ketone group, to the carbon nanoparticle, and the target molecule is bound via an ester, an amide, a thioester, an ether, an amine or a thioether to the bridge molecule.

The target molecules, which are bound to the carbon nanoparticles in the nucleophilic substitution step, may be flexibly chosen depending on the field of application. Monomers, oligomers or copolymers are preferred as target molecules. Especially preferred in this context are epoxides, in particular glycidol.

Also preferred as target molecules are marker molecules, in particular, fluorescent markers for diagnostic applications. In another embodiment, the target molecule is a pharmaceutical agent. Dyes or pigments also may be target molecules. Dyes for capturing light energy (light-capturing pigments) or electrically conductive pigments can be advantageous. Electrical conductors or semiconductors also may be target molecules.

Depending on the particular problem definition, the chemical quality, as well as the size and number of the bridge molecules that are bound to the carbon nanoparticles, are freely selectable. The bridge molecule between the carbonyl group and the nucleofuge may contain a hydrocarbon chain having a length of 2 to 25 carbon atoms. Alternatively, other chain-shaped molecule groups may also be used, such as siloxanes or polyethylene glycols having a chain length of 3 to 25 atoms.

For example, if a large spatial separation is desired between the nanoparticles and the target molecule to produce a preparation, then a long-chain bridge molecule may be selected in conformance therewith.

In the present context, the term preparation signifies a mixture of at least two substances that contains monomers, oligomers and/or copolymers, it being possible for one or more solvents to be part of the preparation. Examples of preparations are adhesives, lacquers, resins or mixtures of copolymers.

For example, if a carbon nanoparticle, that has been modified in accordance with the present invention and is covalently bound via a bridge molecule to a target molecule, is used as a transfection agent for cell biological purposes, then the bridge molecule having a short carbon chain is to be selected.

By carefully selecting the functional groups on the target molecule, the carbon nanoparticles may be made soluble or readily dispersible in conformance with the environment. This is advantageous for purification of the products or when mixing the carbon nanoparticles, that have been modified in accordance with the present invention, into preparations.

In some embodiments carbon nanoparticles, that have been modified and are covalently bound via a bridge molecule to a target molecule, may be used for preparations for medical diagnostics or treatment, as well as for photovoltaics or for electrically conductive or semiconductive components. Carbon nanotubes are preferably used.

The carbon nanoparticles that have been modified may give the preparations special mechanical and chemical properties, such as electrical and thermal conductivity, strength or stability. In the material sciences, such as bonding technology, carbon-reinforced components, for example, a very effective dispersion of the carbon nanoparticles in the particular matrix is often required. It has been said that this may be optimally achieved by the covalent binding of the carbon nanoparticles into the matrix, for example, epoxy resin. In some embodiments the carbon nanoparticles may be used in what are generally known as masterbatches.

The properties of carbon nanoparticles, that have been modified in accordance with embodiments of the present invention, are likewise useful for pharmaceutical purposes. In the first place, carbon nanoparticles may be linked to active agents for treatments that then may act optimally at the effective site of action, by biochemical reaction or as a catalyst for radiation treatments. It was also demonstrated that different carbon nanoparticles may be used as reaction-neutral transfection agents in cell biology. Moreover, the covalent binding to markers, in particular, fluorescent markers, may be carried out, so that diagnostic investigations are made possible.

For example, light-capturing molecules, such as porphyrins or phthalocyanines, when they are bound to electrically conductive carbon nanoparticles that have been modified in accordance with embodiments of the present invention, are able to collect and transmit converted light energy in the form of electric charges. To an increasing degree, carbon nanotubes, that are to replace the established, but costly fullerenes, are being tested in solar technology. The covalent binding of dyes for collecting light or for separating charges offers a practical approach for implementing highly efficient components.

Also, when the carbon nanoparticles that have been modified in accordance with embodiments of the present invention are used in electrical components, the special electrical properties may have important functions either as conductors or semiconductors. For example, in semiconductor technology, one skilled in the art knows that carbon nanotubes function as diodes or as what are generally referred to as nanoswitches, respectively that carbon nanotubes are also used as storage media. In the context of the present invention, it is emphasized here that different components may be covalently linked in a manner that makes economic sense, for example, in order to exploit the possibilities of new connections between metal/semiconductors and carbon nanotubes.

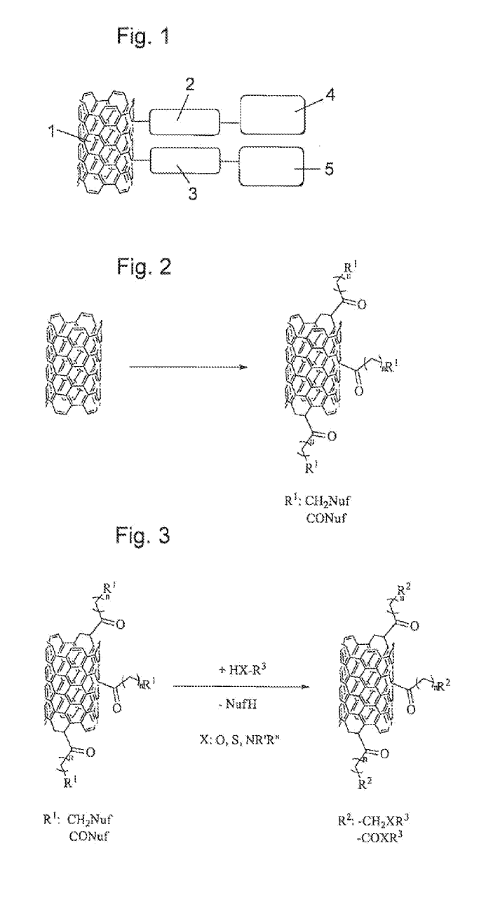

FIG. 1 shows schematically a carbon nanoparticle that has been modified in accordance with an embodiment of the present invention, a carbon nanotube (SWNT) 1 being covalently bound via two different bridge molecules (spacers) 2, 3 to two different target molecules 4, 5. The binding of bridge molecules 2, 3 to carbon nanotubes 1 is carried out in one single reaction step, even when different bridge molecules 2, 3, for example, an omega-substituted carboxylic acid chloride and a cyclic carboxylic acid anhydride are used. In the case of such a mixed derivatization, the binding of different target molecules 4, 5 is also possible, as illustrated in FIG. 1. Thus, the method according to embodiments of the present invention permits the binding to different target molecules 4, 5 which, at the same time, impart different properties to the carbon nanoparticle, such as good dispersibility and collection of light energy.

The reaction sequence in FIG. 2 shows schematically the first method step which is used for binding bridge molecules 2, 3 under Friedel-Crafts conditions to a carbon nanoparticle. The leaving group characterized in FIG. 2 as “Nuf” denotes the nucleofuge which is substituted in the second method step in accordance with FIG. 3 by a nucleophilic functional group on target molecule 4, 5.

The following examples 1-7 correspond to the basic reaction scheme of FIG. 2.

### Example 1

A mixture of 5.4 g (40 mmol) aluminum chloride and 2.4 g (40 mmol) sodium chloride were charged under nitrogen at 120° C. into a 250 mL two-necked flask having a magnetic agitator and reflux condenser that were stored over night at 110° C. The mixture was caused to melt at 160° C. 100 mg of carbon nanotubes (MWNT, Baytubes C150P) were added, as well as succinic anhydride 4.0 g (40 mmol) portionwise for 30 minutes. Upon adding of the anhydride, smoke formation could be observed. The reaction mixture was stirred for two hours at 160° C. and subsequently cooled under nitrogen.

For the decomposition (hydrolysis) of the polyketone aluminum chloride complex, it was treated with ice and 2N hydrochloric acid and subjected to an ultrasound treatment. The suspension was agitated for two hours at 40° C., and filtration of the residual product was carried out using a Büchner filter with suction flask. The acylated carbon nanotubes were washed with water (500 mL). The product was transferred into a centrifugation glass tube and repeatedly slurried in acetone (3 times 25 mL and 2 times 10 mL), centrifuged and decanted. Following drying by exposure to air, a yield of 330 mg acylated carbon nanotubes in the form of black powder was obtained. IR spectroscopy and REM were used to investigate purity.

### Examples 2-7

Example 1 was repeated, except that, this time, the acid chlorides, respectively acid anhydrides indicated in Table 1 were used. The SWNT indicated in Example 3 were produced by pulsed laser evaporation using cobalt and nickel as a catalyst in accordance with the instructions from Carbon 2002, 40, 417-423 by S. Lebedkin, P. Schweiβ, B. Renker, S. Malik, F. Hennrich, M. Neumaier, C. Stoermer, M. M. Kappes.

TABLE 1
Explanations of the synthesis step described in
Example 1 including the acid chlorides, respectively
acid anhydrides indicated in column 2.

| Ex- | Acid chloride, respectively | |
|---|---|---|
| ample | acid anhydride | Yield |
| 2 | glutaric anhydride | from 200 mg MWNT, |
| | (9.58 g, 84 mmol) | 1.53 g of product were obtained |
| 3 | glutaric anhydride | from 50 mg SWNT, |
| | (2.4 g, 21 mmol) | 250 mg of product were obtained |
| 4 | lauroyl chloride (40 mL, | from 1 g MWNT, |
| | 0.17 mol) and | 1.26 g of product were obtained |
| | glutaric anhydride | |
| | (4.8 g, 42 mmol) | |
| 5 | 6-bromohexanoyl chloride | from 120 mg MWNT, |
| | (7.65 mL, 50 mmol) | 249 mg of product were obtained |
| 6 | 4-bromobutyryl chloride | from 120 mg MWNT, |
| | (5.79 mL, 50 mmol) | 197 mg of product were obtained |
| 7 | hexanoyl chloride (48 mL, | from 1 g MWNT, |
| | 0.34 mol) and | 1.38 g of product were obtained |
| | 6-bromohexanoyl chloride | |
| | (12 mL, 80 mmol) | |

Yields of the reactions are listed in column 3.

The reaction sequence in FIG. 3 shows illustratively how the linkage of target molecules 4, 5 in the second method step can proceed. The nucleofuge leaving group (Nuf) in the omega position of a bridge molecule 2, 3 is replaced by a functional nucleophile, denoted by “X” in FIG. 3. The nucleophile is part of a target molecule 4, 5.

The following Examples 8-16 relate to the reaction sequence as illustrated in FIG. 3.

### Example 8

In a 100 mL two-necked flash having a magnetic agitator that was dried over night at 110° C., 54 μl (0.81 mmol) (±)-glycidol were dissolved in 10 mL dichloromethane (over CaH₂). Following the addition of 100 mg acylated carbon nanoparticles, as illustrated under Example 2, 0.22 g (1.15 mmol) EDC HCl (1-ethyl-3-(3-dimethylaminopropyl)carbodiimide hydrochloride) and 40 mg (0.32 mmol) DMAP (N,N-dimethylpyridin) were added. Following agitation of the suspension for 24 hours at room temperature, dichloromethane (50 mL) and water (20 mL) were added. The carbon nanotubes were filtered in a Büchner filter having a suction flask and washed with water (20 mL), saturated sodium chloride solution (20 mL) and water (100 mL). After drying in a vacuum, the product was obtained as black powder at a yield of 50 mg.

### Example 9

The reaction was carried out analogously to Example 8 in a 25 mL two-necked flask with 12.4 mg carbon nanotubes, as illustrated in Example 2, 51.8 mg (0.08 mmol) 5-hydroxymethyl-10,20-bis(undecyl)-21,23H-porphyrin, 17.9 mg (0.09 mmol) EDC HCl and 6 mg (0.05 mmol) DMAP in 3 mL dried dichloromethane. The yield was approximately 2 mg of the black powder.

### Example 10

In a 25 mL two-necked flask having a magnetic agitator and reflux condenser, 100 mg of acylated carbon nanotubes, as illustrated in Example 2, were suspended in 5 mL methanol under nitrogen, and concentrated sulfuric acid (10 drops, 95-97%) was slowly added thereto. The suspension was heated under reflux for two hours. The cooled mixture was evaporated to small volume in a vacuum until dryness, and 25 mL diethyl ether were subsequently added thereto. The carbon nanotubes were filtered in a Büchner filter having a suction flask and washed in each case with 10 mL diethyl ether, saturated sodium hydrogen carbonate solution, water and diethyl ether. The yield was 57 mg of modified carbon nanotubes as black powder.

### Example 11

The reaction was carried out as described in Example 8. In a 25 mL two-necked flask, 25 mg of carbon nanotubes, as illustrated in Example 3, 22 mg (0.034 mmol) of 5-hydroxymethyl-10,20-bis(undecyl)-21,23H-porphyrin, 36 mg (0.18 mmol) EDC HCl and 12 mg (0.1 mmol) of DMAP were reacted with each other in 11 mL of dried dichloromethane. The yield was 11 mg of the black powder.

### Example 12

The reaction was carried out analogously to the description under Example 8. In a 50 mL two-necked flask, 25 mg of acylated carbon nanotubes, as described in Example 3, 96 mg (0.19 mmol) (E)-4-(4-(dimethylamino)styryl)-1-(2-hydroxyethyl)-2,6-diisopropylpyridinium hexafluorophosphate, 550 mg (2.88 mmol) EDC HCl, 94 mg (0.77 mmol) DMAP were reacted under nitrogen in 10 mL dried dichloromethane. The yield of the black powder was 10 mg.

### Example 13

In a 25 mL two-necked flask having a magnetic agitator, acylated carbon nanotubes, as described in Example 3, were suspended under nitrogen in 10 mL of dichloromethane, and 100 mg (0.75 mmol) HOBt H₂O, 180 mg (0.95 mmol) EDC HCl and 160 mg (0.95 mmol) dodecylamine were added thereto. The suspension was agitated for 20 hours at room temperature under nitrogen. The carbon nanotubes were filtered off in a Büchner filter having a suction flask and washed with water (10 mL), 2N HCl (25 mL), saturated sodium hydrogencarbonate solution (25 mL), water (50 mL) and acetone (10 mL). The dried black powder was obtained at a yield of 30 mg.

### Example 14

The reaction was carried out analogously to the description under Example 13. In a 25 mL two-necked flask having a magnetic agitator, 25 mg of acylated carbon nanotubes, as described in Example 3, 124.3 mg (0.25 mmol) (E)-1-(2-aminoethyl)-4-(4-(dimethylamino)styryl)-2,6-diisopropylpyridinium hexafluorophosphate, 45 mg (0.24 mmol) EDC HCl and 25 mg (0.16 mmol) HOBt H₂O were brought to reaction in 10 mL of dichloromethane. The product was obtained as black powder at a yield of 7 mg.

### Example 15

In a 25 mL two-necked flask having a magnetic agitator that was dried over night at 110° C., 20 μl (0.3 mmol) (±)-glycidol and, portionwise, 14 mg (0.35 mmol) sodium hydride (60% suspension in paraffinic oil) were introduced under nitrogen in 2 mL THF (anhydrous, stabilizer-free). The suspension was agitated for one hour at room temperature prior to the addition of 50 mg of acylated carbon nanotubes from Example 5. The black suspension was agitated for another 23 hours at room temperature under nitrogen. 4 mL of saturated ammonium chloride solution were added and the carbon nanotubes were filtered off. The product was washed with water (20 mL) and n-hexane (10 mL). The product was obtained in the form of a black powder at a yield of 20 mg.

### Example 16

The reaction was carried out analogously to the description under Example 15. In a 250 mL two-necked flask having a magnetic agitator that was dried over night at 110° C., 300 mg (7.5 mmol) of sodium hydride (60% suspension in paraffinic oil) were added portionwise to 1.6 mL (19 mmol) (±)-glycidol in 150 mL THF (absolute, without stabilizers), under nitrogen. After one hour at room temperature, 2.25 g of acylated carbon nanotubes, as described in Example 7, were added and agitated at room temperature. The product was obtained as black powder in 2.08 g.

While the invention has been particularly shown and described with reference to preferred embodiments thereof, it will be understood by those skilled in the art that various changes in form and details may be made therein without departing from the spirit and scope of the invention.

### LIST OF REFERENCE NUMERALS

1 carbon nanotubes
2 bridge molecule 1
3 bridge molecule 2
4 target molecule 1
5 target molecule 2

What is claimed is:

1. A method for covalently binding target molecules to carbon nanoparticles via at least one bridge molecule, the method comprising:
a) converting carbon nanoparticles to acyl carbon nanoparticles using a carbonyl compound of at least one bridge molecule in the presence of a Lewis acid under Friedel-Crafts conditions, the acyl carbon nanoparticles including a nucleofuge in the omega position; and
b) covalently binding a target molecule to the acyl carbon nanoparticles via nucleophilic substitution of the nucleofuge in the omega position.

2. The method as recited in claim 1, the carbonyl compound of the at least one bridge molecule is at least one of a carboxylic acid chloride and a carboxylic acid anhydride.

3. The method as recited in claim 1, wherein the carbon nanoparticles are carbon nanotubes.

4. The method as recited in claim 1, wherein the nucleofuge includes a halogen.

5. The method as recited in claim 1, wherein the nucleofuge includes a hydroxyl group of a carboxyl group.

6. The method recited in claim 1, wherein the target molecule has a functional group as a nucleophilic substituent which replaces the nucleofuge during step b), the functional group selected from the group consisting of a hydroxyl group, a thiol group and an amino group.

7. Modified carbon nanoparticles, which are covalently bound via at least one bridge molecule to a target molecule, the modified carbon nanoparticles comprising:
a carbon nano particle;
a target molecule;
at least one bridge molecule bound via a carbonyl group to the carbon nanoparticle, the at least one bridge molecule also bound via a second group to the target molecule, the second group selected from the group consisting of an ester group, a thioester group, an amide group, an ether group, a thioether group and an amine group; and
a chain between the carbon nanoparticle and the target molecule, the chain selected from the group consisting of a hydrocarbon chain, a polyethylene glycol chain and a siloxane chain.

8. Modified carbon nanoparticles as recited in claim 7, wherein the at least one bridge molecule includes at least two different bridge molecules bound to the carbon nanoparticle.

9. Modified carbon nanoparticles as recited in claim 7, wherein the target molecule is a molecule selected from the group consisting of an epoxide, an optically active molecule, a polymer, a pharmaceutical agent, an electrical conductor and a semiconductor.

10. Modified carbon nanoparticles as recited in claim 9, wherein the target molecule includes glycidol.

11. Modified carbon nanoparticles as recited in claim 9, wherein the target molecule is a molecule selected from the group consisting of a porphyrin and a phthalocyanine.

12. A method of using modified carbon nanoparticles that are covalently bound via a bridge molecule to a target molecule, the method including:
preparing the modified carbon nanoparticles, the modified carbon nanoparticles including:
a carbon nano particle,
a target molecule,
at least one bridge molecule bound via a carbonyl group to the carbon nanoparticle, the at least one bridge molecule also bound via a second group to the target molecule, the second group selected from the group consisting of an ester group, a thioester group, an amide group, an ether group, a thioether group and an amine group to the target molecule, and
a chain between the carbon nanoparticle and the target molecule, the chain selected from the group consisting of a hydrocarbon chain, a polyethylene glycol chain and a siloxane chain; and
providing an end product including the modified carbon nanoparticles, the end product being selected from the group consisting of an adhesive, a preparation, a polymer, a medical diagnosis agent, a medical diagnosis treatment agent, and a photovoltaic device.

13. The method as recited in claim 12, wherein the at least one bridge molecule includes at least two different bridge molecules bound to the carbon nanoparticle.

14. The method as recited in claim 12, wherein the target molecule is a molecule selected from the group consisting of an epoxide, an optically active molecule, a polymer, a pharmaceutical agent, an electrical conductor and a semiconductor.

15. The method as recited in claim 14, wherein the target molecule includes glycidol.

16. The method as recited in claim 14, wherein the target molecule is a molecule selected from the group consisting of a porphyrin and a phthalocyanine.
